(19) United States
(12) Patent Application Publication
(10) Pub. No.: US 2019/0092712 A1
(43) Pub. Date: Mar. 28, 2019

(54) CHLOROPROPENE STABILIZATION PROCESS

(71) Applicant: BLUE CUBE IP LLC, Clayton, MO (US)

(72) Inventors: Max Tirtowidjojo, Clayton, MO (US); Susan Campbell, Clayton, MO (US); Greg Stanford, Clayton, MO (US); Jack Kruper, Jr., Clayton, MO (US); Brian Kolthammer, Clayton, MO (US)

(73) Assignee: BLUE CUBE IP LLC, Clayton, MO (US)

(21) Appl. No.: 16/083,223

(22) PCT Filed: Mar. 9, 2017
(86) PCT No.: PCT/US2017/021631

§ 371 Date: Sep. 7, 2018

Related U.S. Application Data
(60) Provisional application No. 62/306,727, filed on Mar. 11, 2016.

(51) Int. Cl. C07C 17/42; B65D 65/42

(52) U.S. Cl. CPC C07C 17/42; B65D 65/42

(57) ABSTRACT

Processes for stabilizing chloropropenes by removing oxygen and/or water and adding substituted phenols as stabilizers.

### FIELD OF THE INVENTION

The present disclosure relates to the stabilization of chloropropenes, including tetrachloropropenes.

### BACKGROUND

Major producers of refrigerant for automobile air conditioning are developing the fourth generation of refrigerants with 2,3,3,3-tetrafluororopene (HFO-1234yf) as a replacement for CFC-134a. This hydrofluoro-olefin has properties that meet European requirements of low global warming potential and no impact on the ozone layer. The most cost efficient feedstock for the production of HFO-1234yf has been identified to be 1,1,2,3-tetrachloropropene (TCPE). Chlorinated organic compounds, including chloropropenes, can degrade over time upon contact with air, humidity, heat, light, and/or metal. Degradation products include acids and oxidation by-products. Thus, there is a need to stabilize chloropropenes and prevent formation of these degradation products.

### BRIEF DESCRIPTION OF THE DRAWINGS

FIG. 1 presents a comparison for samples stored under nitrogen or stored under moist air. The GC area % of TCPE for each condition is plotted as a function of time.

### SUMMARY

One aspect of the present disclosure encompasses a process for stabilizing a chloropropene. The process comprises purging a system containing the chloropropene with an inert gas. The process further comprises adding a stabilizing amount of at least one substituted phenol, wherein the stabilizing amount substituted phenol is at least 1 ppm by weight of the chloropropene.

Another aspect of the present disclosure provides a composition comprising a tetrachloropropene and a stabilizing amount of 2-isopropyl-5-methylphenol.

Other aspects and iteration of the disclosure are described in more detail below.

### DETAILED DESCRIPTION

The present disclosure provides processes for stabilizing chloropropenes, including tetrachloropropenes. Stabilization of chloropropenes reduces or eliminates the formation of degradation products and/or oxidation products during transport and/or storage of the compounds. Importantly, the stabilized chloropropenes are not degraded when exposed to elevated temperatures and/or humid environments.

### I. Process for Stabilizing Chloropropenes

One aspect of the present disclosure provides processes for stabilizing chloropropenes. The process comprises purging a system containing the chloropropene with an inert gas, and adding a stabilizing amount of at least one substituted phenol to the system, wherein the stabilizing amount of the substituted phenol is at least 1 ppm by weight of the chloropropene.

### (a) Purging the System

The first step of the process comprises purging a system containing the chloropropene with an inert gas to reduce the level of oxygen and/or moisture in the system.

(ii) Chloropropene

Chloropropenes include compounds having one, two, three, four, five, or six chlorine atoms, which are referred to as mono-, di-, tri-, tetra-, penta-, or hexa-chloropropenes, respectively. Suitable chloropropenes include monochloropropenes, such as E-1-chloropropene, Z-1-chloropropene, 2-chloropropene, and 3-chloropropene, dichloropropenes, such as 1,1-dichloropropene, E-1,2-dichloropropene, Z-1,2-dichloropropene, E-1,3-dichloropropene, Z-1,3-dichloropropene, 2,3-dichloropropene, and 3,3-dichloropropene, trichloropropenes, such as 1,1,2-trichloropropene, 1,1,3-trichloropropene, E-1,2,3-trichloropropene, Z-1,2,3-trichloropropene, E-1,3,3-trichloropropene, Z-1,3,3-trichloropropene, 2,3,3-trichloropropene, and 3,3,3-trichloropropene, tetrachloropropenes, such as 1,1,2,3-tetrachloropropene, 1,1,3,3-tetrachloropropene, E-1,2,3,3-tetrachloropropene, Z-1,2,3,3-tetrachloropropene, E-1,3,3,3-tetrachloropropene, Z-1,3,3,3-tetrachloropropene, and 2,3,3,3-tetrachloropropene, pentachloropropenes, such as 1,1,2,3,3-pentachloropropene, 1,1,3,3,3-pentachloropropene, E-1,2,3,3,3-pentachloropropene, and Z-1,2,3,3,3-pentachloropropene, hexachloropropene, or combinations thereof. In specific embodiments, the chloropropene may be a tetrachloropropene. In exemplary embodiments, the chloropropene may be 1,1,2,3-tetrachloropropene (TCPE).

In some embodiments, the chloropropene may be treated to remove water and/or oxygenates. As used herein, the term “oxygenate” refers to a hydrocarbon substituted with at least one oxygen atom. The treatment may comprise contact with a molecular sieve and/or contact with an alkylene oxide. Non-limiting examples of suitable molecular sieves include alumina (i.e., aluminum oxide), active carbon (also called activated charcoal), clays (e.g., bentonites, kaolinites, nontronites, montmorillonites, pyrophyllites, saponites, sauconites, vermiculites, and the like), silica gel, zeolites, or combinations thereof. In specific embodiments, the molecular sieve may be alumina. The amount of molecular sieve that is contacted with the chloropropene will vary but those skilled in the art can readily determine the appropriate amount. Interaction between an alkylene oxide and water leads to formation of the corresponding alkylene glycol. Suitable alkylene oxides include ethylene oxide, propylene oxide, butylene oxide, and so forth. In general, the amount of alkylene oxide that is contacted with the chloropropene is less that about 2000 ppm, less than about 1000 ppm, less than about 500 ppm, or less than about 100 ppm.

In certain embodiments, the chloropropene may be substantially free of water and/or oxygenates. As used herein, the phrase “substantially free” generally means there is no or very little water that can be detected using standard detection means. For example, the chloropropene may contain less than about 1%, less than about 0.5%, less than about 0.1%, less than about 0.01%, less than about 0.001%, or less than about 0.0001% of water and/or oxygenates.

(ii) System

The system which contains the chloropropene can and will vary. In some embodiments, the system may be a container. Non-limiting examples of suitable containers include tanks, barrels, drums, intermediate bulk containers (IBCs), carboys, reaction vessels, storage vessels, and the like. In certain embodiments, the container may be a sealable container and/or the container may be a light blocking container. In various embodiments, the container may be a metal container. The metal may be a single metal or an alloy of various metals. Suitable metals include, without limit, stainless steel, carbon steel, nickel alloys, zinc aluminum alloys, aluminum, tin, brass, or combinations thereof. In specific embodiments, the metal may be stainless steel, carbon steel, or a monel metal. A monel metal is a nickel alloy, primarily composed of nickel (up to 67%) and copper, with small amounts of iron, manganese, carbon, and silicon. In certain embodiments, the container may be lined with a polymeric coating. The polymeric coating may be a phenolic coating or an epoxy coating. In exemplary embodiments, the coating may be a PLASITE® coating (available from Carboline Company).

(iii) Purging the System

The process comprises purging the system with an inert gas. The inert gas may be nitrogen, argon, helium, neon, xenon, or a mixture thereof. In specific embodiments, the inert gas may be nitrogen.

The amount of inert gas used to purge the system can and will vary depending upon a variety of factors, including, e.g., the volume of the system. Similarly the flow rate of the inert gas used to purge the system can and will vary depending upon various factors, such as, e.g., the configuration of the system. Those skilled in the art are familiar with means for determining these parameters. In general, the purging step will proceed for at least about 5 minutes at ambient temperature. In some embodiments, the purging step may proceed for at least about 10 minutes, at least about 30 minutes, at least about 60 minutes, at least about 120 minutes, or longer than about 120 minutes at ambient temperature.

After purging, the system and the chloropropene in the system may be substantially free of oxygen. As used herein, the phrase “substantially free” generally means there is no or very little oxygen that can be detected using standard detection means. For example, the chloropropene may contain less than about 1%, less than about 0.5%, less than about 0.1%, less than about 0.01%, less than about 0.001%, or less than about 0.0001% of oxygen.

### (b) Adding the Stabilizer

The process further comprises adding a stabilizing amount of at least one substituted phenol to the purged system. The substituted phenol stabilizes the chloropropene by inhibiting oxidation and/or degradation of the chloropropene. The substituted phenol comprises at least one substituent chosen from chloro, nitro, nitroso, alkoxy, hydroxy, amino, alkylamino, keto, alkanoyl, alkyl, substituted alkyl, alkenyl, substituted alkenyl, aryl, or substituted aryl.

Non-limiting examples of suitable substituted phenols include 2-isopropyl-5-methylphenol (also called isopropyl-m-cresol, hydroxy cymene, or thymol), p-methoxyphenol, p-tert-amylphenol, p-sec-butylphenol, 2,4-dimethyl-6-tert-butylphenol, 2-methoxyhydroquinone, butylated hydroxy anisole, butylated hydroxy toluene, 4-nitrophenol, m-cresol, o-cresol, p-cresol, 2-chloro-m-cresol, p-chlorocresol, hydroquinone, methylhydroquinone, 2,3-dimethylhydroquinone, tert-butyl hydroquinone, 2,4-di-tert-butylphenol, 2,6-di-tert-butylphenol, 2,6-di-tert-butyl-4-methylphenol, 2,6-di-tert-butyl-4-ethylphenol, 2,4-dimethyl-6-tert-butylphenol, 2,6-di-tert-butyl-4-(dimethylaminomethyl)phenol, 4,4′-thiobis(2-methyl-6-tert-butylphenol), 4,4′-thiobis(3-methyl-6-tert-butylphenol), 2,2′-thiobis(4-methyl-6-tert-butylphenol), 4,4′-methylenebis(2,6-di-tert-butylphenol), 4,4′-bis(2,6-di-tert-butylphenol), 2,2′-methylenebis(6-tert-butyl-4-ethylphenol), 2,2′-methylenebis(6-tert-butyl-4-methylphenol), 4,4-butylidenebis(3-methyl-6-tert-butylphenol), 4,4-isopropylidenebis(2,6-di-tert-butylphenol), 2,2′-methylenebis(4-methyl-6-nonylphenol), 2,2′-isobutylidenebis(4,6-dimethylphenol), 2,2′-methylenebis(4-methyl-6-cyclohexylphenol), 2,2′-ethylidene-bis(4,6-di-tert-butylphenol), 2,6-di-tert-butyl-4-(N,N′-dimethyl-aminomethyl)-phenol, 2-hydroxybenzophenone, 2,4-dihydroxybenzophenone, 2,2′-dihydroxy-4,4′-dimethoxybenzophenone, 2,2′-dihydroxy-4-methoxybenzophenone, 4-allyloxy-2-hydroxybenzophenone, 5-chloro-2-hydroxybenzophenone, 5-chloro-2-hydroxy-4-methylbenzophenone, 2-(2H-benzotriazol-2-yl)-4,6-bis(1-methyl-1-phenylethyl)phenol, 2-(2H-benzotriazol-2-yl)-4,6-di-tert-pentylphenol, 2-(2H-benzotriazol-2-yl)-6-dodecyl-4-methylphenol, 2-[3-(2H-benzotriazol-2-yl)-4-hydroxyphenyl]ethyl methacrylate, 2-(2H-benzotriazol-2-yl)-4-methyl-6-(2-propenyl)phenol, 2-(4-benzoyl-3-hydroxyphenoxy)ethyl acrylate, 2-tert-butyl-6-(5-chloro-2H-benzotriazol-2-yl)-4-methylphenol, 2,4-di-tert-butyl-6-(5-chloro-2H-benzotriazol-2-yl)phenol, 3′,5′-dichloro-2′-hydroxyacetophenone, 2′,4′-dihydroxy-3′-propylacetophenone, 2-(4,6-diphenyl-1,3,5-triazin-2-yl)-5-[(hexyl)oxy]-phenol, 2-ethylhexyl salicylate, 5,5′-methylenebis(2-hydroxy-4-methoxybenzophenone), pentaerythritol tetrakis(3,5-di-tert-butyl-4-hydroxyhydrocinnamate), 1,3,5-trimethyl-2,4,6-tris(3,5-di-tert-butyl-4-hydroxybenzyl)benzene, tris(4-tert-butyl-3-hydroxy-2,6-dimethylbenzyl)isocyanurate, or combinations thereof.

In some embodiments, the substituted phenol may be 2-isopropyl-5-methylphenol, p-methoxyphenol, p-tert-amylphenol, p-sec-butylphenol, 2,4-dimethyl-6-tert-butylphenol, 2-methoxyhydroquinone, butylated hydroxy anisole, butylated hydroxy toluene, or combinations thereof. In exemplary embodiments, the substituted phenol may be 2-isopropyl-5-methylphenol.

A stabilizing amount of the substituted phenol refers to an amount that reduces and/or prevents degradation and/or oxidation of the chloropropene and formation of degradation products such as, e.g., hydrochloric acid, phosgene, chloroform, chloroethanes, chloropropanones, other oxidation products, oligomers, isomers, and the like. In general, the stabilizing amount is at least 1 ppm by weight of the chloropropene. In some embodiments, the amount of substituted phenol added to the system containing the chloropropene may range from about 1 ppm to about 10,000 ppm by weight of the chloropropene. In other embodiments, the amount of substituted phenol added to the system may range from about 1 ppm to about 1000 ppm by weight of the chloropropene. In still other embodiments, the amount of substituted phenol added to the system may range from about 1 ppm to about 100 ppm by weight of the chloropropene. In various embodiments, the amount of amount of substituted phenol added to the system may range from about 1 ppm to about 3 ppm, from about 3 ppm to about 10 ppm, from about 10 ppm to about 30 ppm, from about 30 ppm to about 100 ppm, from about 100 ppm to about 300 ppm, from about 300 ppm to about 1000 ppm, from about 1000 ppm to about 3000 ppm, or from about 3000 ppm to about 10,000 ppm.

### (c) Optional Steps

In some embodiments, the process may further comprise, after adding the substituted phenol, padding or blanketing the system with an inert gas, such that the stabilized chloropropene is overlaid with a pad or blanket of inert gas. The inert gas may be nitrogen, argon, helium, neon, xenon, or a mixture thereof. In specific embodiments, the inert gas may be nitrogen. After padding the system, the system may be sealed using sealing system that is appropriate for the system.

In other embodiments, the process may further comprise, after step b), transferring the chloropropene from the system to a storage container, padding the storage container with an inert gas (as detailed above), and sealing the storage container with a suitable sealing system. Suitable storage containers include tanks, barrels, drums, intermediate bulk containers (IBCs), and the like.

### (d) Stabilized Chloropropene

At the end of the process, the chloropropene is stabilized. That is, the stabilized chloropropene is less susceptible to degradation than chloropropene that has not been stabilized. In particular, even upon exposure to air, humidity, heat, light, and/or time, the stabilized chloropropene is less susceptible to degradation than unstabilized chloropropene. Thus, the stabilized chloropropene generally has lower levels of degradation products (e.g., hydrochloric acid, phosgene, chloroform, chloroethanes, chloropropanones, other oxidation products, oligomers, isomers, and the like) than chloropropene that has not been stabilized.

The stabilized chloropropene generally is stable (and can be stored) for at least 1 month, at least 3 months, at least 6 months, at least 9 month, at least 12 months, at least 18 months, at least 24 months, at least 36 month, or at least 48 months. The storage temperature is typically at ambient conditions. In some embodiments, the storage temperature may range from about 0° C. to about 50° C., from about 10° C. to about 30° C., or from about 15° C. to about 25° C. In other embodiments, the temperature is less than or equal to 40° C.

In specific embodiments, the stabilized chloropropene comprises a tetrachloropropene and a stabilizing amount of 2-isopropyl-5-methylphenol. In exemplary embodiments, the tetrachloropropene may be 1,1,2,3-tetrachloropropene. The amount of 2-isopropyl-5-methylphenol present in the stabilized tetrachloropropene may range from about 1 ppm to about 10,000 ppm, from about 1 ppm to about 1000 ppm, or from about 1 ppm to about 100 ppm by weight of the tetrachloropropene. In some embodiments, the amount of 2-isopropyl-5-methylphenol present in the stabilized tetrachloropropene may be about 5 ppm, about 10 ppm, about 15 ppm, about 20 ppm, about 25 ppm, about 30 ppm, about 40 ppm, about 45 ppm, about 50 ppm, about 200 ppm, about 200 ppm, about 500 ppm, about 1000 ppm, about 5000 ppm, about 10,000 ppm, or any intervening value.

In general, the stabilized chloropropene is substantially free of water, oxygenates, and/or oxygen, as defined above.

### Definitions

Unless defined otherwise, all technical and scientific terms used herein have the meaning commonly understood by a person skilled in the art to which this invention belongs.

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “alkyl” as used herein describes groups containing from one to thirty carbon atoms in the principal chain. They may be straight or branched chain or cyclic and include methyl, ethyl, propyl, isopropyl, butyl, hexyl and the like.

The term “alkenyl” as used herein describes groups containing from two to thirty carbon atoms in the principal chain and further comprising at least one carbon-carbon double bond. They may be straight or branched chain or cyclic and include ethenyl, propenyl, isopropenyl, butenyl, isobutenyl, hexenyl, and the like.

The term “alkoxide” or “alkoxy” as used herein is the conjugate base of an alcohol. The alcohol may be straight chain, branched, cyclic, and includes aryloxy compounds.

The term “aryl” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The term “substituted” with regard to alkyl, alkenyl, and aryl refers to said moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

### EXAMPLES

The following examples are included to demonstrate preferred embodiments of the invention. It should be appreciated by those of skill in the art that the techniques disclosed in the examples represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth is to be interpreted as illustrative and not in a limiting sense

### Examples 1-3: Aging Studies of 1,1,2,3-Tetrachloropropene

Samples of 1,1,2,3-tetrachloropropene (TCPE) (99% pure) were used as received (i.e., untreated) or treated with alumina to remove water and oxygenate impurities (treated). The samples were purged with nitrogen upon receipt and transferred to an inert atmosphere glove box (Vacuum Atmospheres) prior to use. The samples were stored under various conditions (e.g., inert atmosphere, humid air, etc.), and incubated at 40° C. for 6 months (i.e., 182 days). Samples were removed biweekly and analyzed by GC (HP 6890 GC and ChemStation-based software) for TCPE content. Hexachloroethane (HCE) was chosen as an internal standard to test for the decomposition of TCPE during storage.

### Example 1: Storage of 1,1,2,3-Tetrachloropropene Under Inert Atmosphere

The first set of experiments was carried out in a Vacuum Atmospheres DLX-002-S-P inert atmosphere box operated under a continuous nitrogen purge (20 standard cubic foot per hour (scfh)) with the vent purge scrubbed with alumina prior to release. The oxygen content in the box was monitored by an Illinois Instruments Model 911 oxygen meter. The environment in the box contained less than 0.2 ppmv oxygen for the duration of the experiment.

Aliquots (30 mL) of TCPE (untreated and treated) were transferred to 40 mL Fisher Scientific I-Chem vials in the dry box. Samples were either used as is or modified according to experimental design shown in Table 1. Due to the duration of the experiment (6 months), each test was carried out in duplicate (specimen A and B) to allow for adequate volumes for the number of samples and to yield some understanding of reproducibility of the observations. The tests for experiment 1 included observations of the presence of stainless steel (SS) or Plasite-coated steel coupons during aging. In addition, the impact of the presence of small amounts of a stabilizer, THYMOL (2-isopropyl-5-methylphenol; Fisher Scientific), on the aging was also studied. (A stock solution of THYMOL in TCPE was prepared by diluting 45 mg of solids to a total volume of 100 mL. Tests with 10 ppmw (1 mL) and 20 ppmw (2 mL) incorporated the aliquot in the 30 mL total volume of the test mixture.)

Once assembled and capped, the sample vials were place in a J-Kem RBR-40 heated reaction block mounted on a LabLine Orbit Shaker. The reaction block was heated to 40° C. using a J-Kem Model 150 temperature Controller and the block shaken at a rate of 125 rpm.

TABLE 1
Experimental design for TCPE aging study in N2

| | temp | | | |
|---|---|---|---|---|
| run # | (° C.) | MoC/Coup | TCPE | inhibitor |
| 1 | 40 | none | untreated | no |
| 2 | 40 | none | untreated | Thymol 10 ppmw |
| 3 | 40 | SS | untreated | No |
| 4 | 40 | SS | untreated | Thymol 10 ppmw |
| 5 | 40 | plasite | untreated | No |
| 6 | 40 | plasite | untreated | Thymol 10 ppmw |
| 7 | 40 | none | untreated | Thymol 20 ppmw |
| 8 | 40 | SS | untreated | Thymol 20 ppmw |
| 9 | 40 | plasite | untreated | Thymol 20 ppmw |
| 10 | 40 | none | treated | No |
| 11 | 40 | plasite | treated | No |
| 12 | 40 | plasite | treated | Thymol 10 ppmw |
| 13 | 40 | plasite | treated | Thymol 20 ppmw |

The samples prepared and stored under nitrogen allowed for the observation of the effect of temperature, and the effect of the presence of stainless steel surfaces on the stability of TCPE. On the assumption that stainless steel may accelerate decomposition, Plasite-coated steel coupons were also tested to check for compatibility with TCPE and whether this coating eliminated decomposition. During the gentle shaking of the samples in the rotary shaker, the Plasite coating eroded from the coupon ion all samples in which they were present. This erosion led to the development of some murkiness in the relevant samples (5, 6, 9, 11, 12 and 13). The levels of TCPE and HCE in each sample are presented in Tables 2 and 3, respectively.

TABLE 2
TCPE Assay

| Time (Days) | Run 1 | Run 2 | Run 3 | Run 4 | Run 5 | Run 6 | Run 7 | Run 8 | Run 9 | Run 10 | Run 11 | Run 12 | Run 13 |
|---|---|---|---|---|---|---|---|---|---|---|---|---|---|
| 0 | 99.4 | 99.2 | 99.2 | 99.1 | 99.2 | 99.0 | 98.7 | 98.7 | 98.7 | 99.3 | 99.3 | 99.0 | 98.9 |
| 14 | 99.4 | 99.2 | 99.2 | 99.1 | 99.2 | 99.1 | 98.8 | 98.7 | 98.7 | 99.4 | 99.3 | 99.2 | 98.9 |
| 42 | 99.2 | 99.2 | 99.2 | 99.1 | 99.3 | 99.2 | 98.9 | 98.8 | 98.7 | 99.4 | 99.4 | 99.2 | 98.9 |
| 56 | 99.3 | 99.1 | 99.2 | 99.1 | 99.2 | 99.1 | 98.8 | 98.7 | 98.6 | 99.3 | 99.4 | 99.2 | 98.8 |
| 70 | 99.1 | 99.2 | 99.2 | 99.1 | 99.2 | 99.1 | 98.7 | 98.7 | 99.0 | 99.3 | 99.3 | 99.3 | 98.9 |
| 84 | 99.3 | 98.4 | 98.3 | 98.3 | 99.1 | 99.0 | 98.7 | 98.7 | 98.6 | 99.4 | 99.3 | 99.2 | 98.8 |
| 98 | 99.3 | 99.1 | 99.0 | 99.1 | 98.8 | 98.9 | 98.6 | 98.6 | 98.5 | 99.3 | 99.3 | 99.2 | 98.7 |
| 112 | 99.3 | 99.1 | 99.2 | 99.1 | 99.2 | 99.0 | 98.6 | 98.6 | 98.3 | 99.2 | 99.3 | 99.1 | 98.4 |
| 126 | 99.1 | 99.2 | 99.2 | 98.8 | 99.2 | 99.0 | 98.4 | 98.3 | 98.3 | 99.3 | 99.4 | 98.9 | 98.4 |
| 140 | 99.2 | 98.9 | 99.1 | 99.0 | 99.2 | 99.0 | 98.5 | 98.3 | 98.2 | 99.2 | 99.3 | 98.9 | 98.4 |
| 154 | 99.2 | 98.9 | 99.1 | | 99.1 | 98.8 | 98.3 | 98.2 | 98.2 | 99.3 | 99.3 | 98.9 | 98.3 |
| 168 | 99.4 | 99.0 | 99.3 | 98.9 | 99.3 | 99.0 | 98.5 | 98.5 | 98.5 | 99.3 | 99.3 | 99.0 | 98.6 |
| 182 | 99.2 | 98.7 | 99.1 | 98.8 | 99.1 | 98.8 | 98.2 | 98.1 | 98.1 | 99.3 | 99.4 | 99.1 | 98.6 |

TABLE 3
HCE Assay

| Time (Days) | Run 1 | Run 2 | Run 3 | Run 4 | Run 5 | Run 6 | Run 7 | Run 8 | Run 9 | Run 10 | Run 11 | Run 12 | Run 13 |
|---|---|---|---|---|---|---|---|---|---|---|---|---|---|
| 0 | 0.274 | 0.273 | 0.273 | 0.274 | 0.275 | 0.275 | 0.286 | 0.273 | 0.276 | 0.280 | 0.276 | 0.281 | 0.283 |
| 14 | 0.273 | 0.275 | 0.274 | 0.281 | 0.273 | 0.271 | 0.272 | 0.277 | 0.273 | 0.283 | 0.278 | 0.277 | 0.278 |
| 42 | 0.276 | 0.278 | 0.276 | 0.274 | 0.274 | 0.274 | 0.273 | 0.273 | 0.277 | 0.281 | 0.281 | 0.278 | 0.280 |
| 56 | 0.273 | 0.277 | 0.276 | 0.277 | 0.276 | 0.276 | 0.273 | 0.275 | 0.275 | 0.283 | 0.280 | 0.285 | 0.281 |
| 70 | 0.277 | 0.276 | 0.288 | 0.278 | 0.278 | 0.277 | 0.275 | 0.277 | 0.292 | 0.281 | 0.288 | 0.289 | 0.281 |
| 84 | 0.282 | 0.280 | 0.457 | 0.270 | 0.279 | 0.276 | 0.276 | 0.275 | 0.275 | 0.280 | 0.281 | 0.282 | 0.276 |
| 98 | 0.282 | 0.280 | 0.277 | 0.275 | 0.278 | 0.278 | 0.275 | 0.292 | 0.277 | 0.284 | 0.286 | 0.281 | 0.281 |
| 112 | 0.283 | 0.277 | 0.279 | 0.276 | 0.278 | 0.280 | 0.291 | 0.278 | 0.281 | 0.281 | 0.284 | 0.281 | 0.277 |
| 126 | 0.278 | 0.283 | 0.281 | 0.275 | 0.288 | 0.277 | 0.275 | 0.277 | 0.274 | 0.285 | 0.280 | 0.280 | 0.279 |
| 140 | 0.283 | 0.289 | 0.279 | 0.280 | 0.282 | 0.279 | 0.282 | 0.277 | 0.275 | 0.285 | 0.282 | 0.280 | 0.277 |
| 154 | 0.281 | 0.283 | 0.283 | | 0.278 | 0.276 | 0.275 | 0.281 | 0.275 | 0.283 | 0.283 | 0.282 | 0.281 |
| 168 | 0.280 | 0.344 | 0.282 | 0.283 | 0.281 | 0.284 | 0.283 | 0.279 | 0.280 | 0.287 | 0.293 | 0.291 | 0.283 |
| 182 | 0.281 | 0.278 | 0.285 | 0.279 | 0.281 | 0.277 | 0.276 | 0.286 | 0.278 | 0.281 | 0.290 | 0.283 | 0.281 |

The first set of runs show the impact of a higher temperature (40° C.) on the storage and handling (agitation) of the TCPE. Samples of TCPE that were maintained under inert atmosphere at 40° C. showed no signs of degradation over a period of 24 weeks. Additionally, there was no measurable difference between untreated TCPE (no coupon and no inhibitor; Run 1) and treated TCPE (no coupon and no inhibitor; Run 10).

### Example 2: Storage of 1,1,2,3-Tetrachloropropene in Humid Air

The second set of experiments was carried out in a Fume Hood with materials frequently exposed to ambient air. Aliquots (30 mL) of untreated TCPE were transferred to 40 mL Fisher Scientific I-Chem vials in the dry box. Samples were either used as is or modified according to the experimental table as shown in Table 4 below. Due to the duration of the experiment (6 months), each test was carried out in duplicate (specimen A and B) to allow for adequate volumes for the number of samples and to yield some understanding of reproducibility of the observations. The vials were removed from the dry box and exposed to air. Subsequently, the vials were placed in racks in a Fisher Isotemp 205 water bath operating at 40° C. with the vials submerged to the liquid level inside the vials. During sampling for GC analysis, the materials were exposed to the humid air above the water bath to simulate exposure to ambient storage conditions. No attempt was made to protect the aging TCPE from air or moisture.

TABLE 4
Experimental design for TCPE aging study in ambient air

| | temp | | | |
|---|---|---|---|---|
| run # | (° C.) | MoC/Coup | TCPE | inhibitor |
| 14 | 40 | plasite | untreated | no |
| 15 | 40 | plasite | untreated | Thymol 10 ppmw |
| 16 | 40 | none | untreated | No |
| 17 | 40 | none | untreated | Thymol 20 ppmw |
| 18 | 40 | plasite | untreated | Thymol 20 ppmw |

This set of runs allowed for the observation of the effect of temperature and of air on the stability of the TCPE. In this set, only untreated TCPE was studied, and the presence of only coated coupons was established. This limited set does however also include the impact of the THYMOL stabilizer on storage under air. The results are presented in Tables 5 and 6 below.

TABLE 5
TCPE Assay

| Time (Days) | Run 14 | Run 15 | Run 16 | Run 17 | Run 18 |
|---|---|---|---|---|---|
| 0 | 99.1 | 99.0 | 99.1 | 98.6 | 98.5 |
| 14 | 98.9 | 98.8 | 98.9 | 98.4 | 98.4 |
| 42 | 98.9 | 99.0 | 99.0 | 98.6 | 98.5 |
| 56 | 98.9 | 99.1 | 98.9 | 98.7 | 98.6 |
| 70 | 98.9 | 99.0 | 99.0 | 98.6 | 98.7 |
| 84 | 98.8 | 98.9 | 98.8 | 98.8 | 99.3 |
| 98 | 98.7 | 99.0 | 98.9 | 98.4 | 98.5 |
| 112 | 98.7 | 98.8 | 98.7 | 98.3 | 98.3 |
| 126 | 98.6 | 98.9 | 98.9 | 98.3 | 98.4 |
| 140 | 98.4 | 98.8 | 98.6 | 98.3 | 98.6 |
| 154 | 98.5 | 98.9 | 98.8 | 98.2 | 98.7 |
| 168 | 98.3 | 99.1 | 98.5 | 98.6 | |
| 182 | 98.5 | 99.2 | 98.9 | 98.8 | 98.8 |

TABLE 6
HCE Assay

| Time (Days) | Run 14 | Run 15 | Run 16 | Run 17 | Run 18 |
|---|---|---|---|---|---|
| 0 | 0.278 | 0.276 | 0.275 | 0.273 | 0.271 |
| 14 | 0.274 | 0.273 | 0.273 | 0.273 | 0.273 |
| 42 | 0.275 | 0.277 | 0.274 | 0.279 | 0.270 |
| 56 | 0.276 | 0.276 | 0.280 | 0.273 | 0.282 |
| 70 | 0.279 | 0.277 | 0.274 | 0.282 | 0.275 |
| 84 | 0.279 | 0.274 | 0.273 | 0.276 | 0.281 |
| 98 | 0.282 | 0.275 | 0.277 | 0.276 | 0.274 |
| 112 | 0.275 | 0.278 | 0.276 | 0.274 | 0.272 |
| 126 | 0.277 | 0.276 | 0.284 | 0.274 | 0.274 |
| 140 | 0.276 | 0.276 | 0.278 | 0.272 | 0.279 |
| 154 | 0.279 | 0.276 | 0.280 | 0.274 | 0.276 |
| 168 | 0.281 | 0.290 | 0.286 | 0.280 | 0.284 |
| 182 | 0.280 | 0.275 | 0.287 | 0.280 | 0.274 |

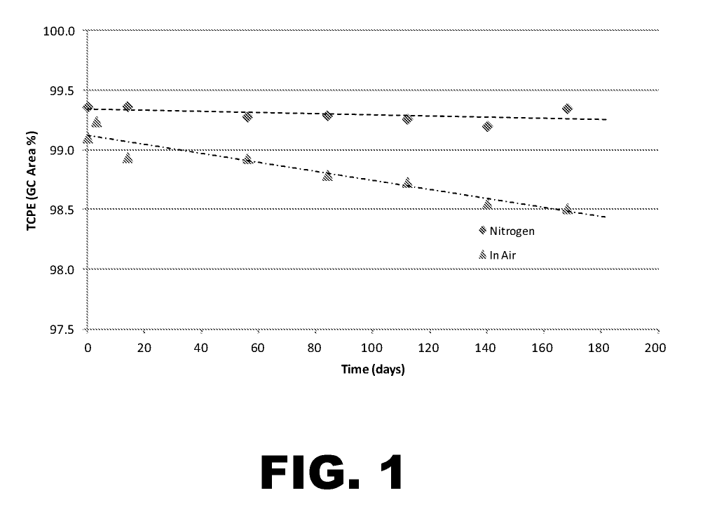

In particular, Run 16 was a sample that contained untreated TCPE alone and the GC data show a slight change at the later stages of the experiment. Although the average values for TCPE don't show significant difference, the standard deviation is greater for the set relative to the standard results. More importantly, a difference plot of TCPE concentrations for Run 16 with Run 1 begins to show a trend (all values negative and increasing in magnitude) after about week 12, indicating a loss in TCPE is occurring. Even through week 26 though, only a small loss (<1% absolute) in TCPE content is noted. This result is reinforced by the observations of Run 14, a sample containing a coated coupon and shaken in air. The same change in the difference plot (Run 14 versus Run 1) appears at about week 12 and the trend to increasing differences continues to week 26. Again, even at week 26, the loss in TCPE is only about 1% absolute. The results for runs 15 (coated coupon, 10 ppmw THYMOL), 18 (coated coupon, 20 ppmw THYMOL) and 17 (no coupon, 20 ppmw THYMOL) are instructive. No loss in TCPE concentration is observed and no trend in difference plots versus the standards is observed. An example of the inference of the difference plots is shown in FIG. 1 where a comparison of the assay results for a pair of nitrogen/air samples is presented.

### Example 3: Storage of 1,1,2,3-Tetrachloropropene in the Presence of Carbon Steel

A third set of experiments was initiated at week 14 when observations indicated that TCPE aging at 40° C. was slow. This last set included four conditions to test the impact of the presence of coupons of carbon steel. The sample conditions are described in Table 7.

TABLE 7
Experimental design for TCPE aging study
in the presence of C-steel coupon

| | temp | | | |
|---|---|---|---|---|
| run # | (° C.) | MoC/Coup | TCPE | inhibitor |
| 19 | 40 | carbon steel under N2 | untreated | no |
| 20 | 40 | carbon steel under N2 | untreated | Thymol 10 ppmw |
| 21 | 40 | carbon steel under air | untreated | No |
| 22 | 40 | carbon steel under air | untreated | Thymol 10 ppmw |

A 12 week series of experiments with carbon steel coupons showed that the presence of these had no impact on the stability of the TCPE under an inert atmosphere. As in Runs 1, 3, and 5, the TCPE and HCE concentrations did not vary significantly over the duration of the run. For the tests with carbon steel in samples shaken under air, no change in TCPE was observed by the end of the 12 weeks. The levels of TCPE and HCE in each sample are presented in Tables 8 and 9, respectively.

TABLE 8
TCPE Assay

| | | | | | TCPE- | TCPE- |
|---|---|---|---|---|---|---|
| Time | Run | Run | Run | Run | Untreated- | Untreated- |
| (days) | 19 | 20 | 21 | 22 | 1209 | 0110 |
| 2 | 99.0 | 98.6 | 99.0 | 98.5 | 99.0 | 99.0 |
| 9 | 98.9 | 98.8 | 98.9 | 98.6 | | |
| 16 | 99.0 | 98.6 | 98.9 | 98.6 | 99.1 | 99.1 |
| 30 | 99.2 | 98.8 | 98.9 | 98.9 | 99.2 | 99.2 |
| 44 | 99.1 | 98.9 | 98.8 | 98.8 | 99.2 | 99.1 |
| 58 | 99.1 | 98.7 | 98.9 | 99.1 | 99.1 | 99.1 |
| 72 | 99.1 | 98.8 | 98.9 | 99.0 | 99.1 | 99.2 |
| 86 | 99.2 | 99.1 | 98.7 | 99.3 | 99.2 | 99.3 |

TABLE 9
HCE Assay

| | | | | | TCPE- | TCPE- |
|---|---|---|---|---|---|---|
| Time | Run | Run | Run | Run | Untreated- | Untreated- |
| (days) | 19 | 20 | 21 | 22 | 1209 | 0110 |
| 2 | 0.276 | 0.275 | 0.275 | 0.276 | 0.275 | 0.240 |
| 9 | 0.278 | 0.285 | 0.276 | 0.280 | | |
| 16 | 0.278 | 0.279 | 0.278 | 0.277 | 0.283 | 0.240 |
| 30 | 0.279 | 0.279 | 0.280 | 0.277 | 0.282 | 0.244 |
| 44 | 0.279 | 0.282 | 0.278 | 0.279 | 0.278 | 0.245 |
| 58 | 0.277 | 0.275 | 0.279 | 0.275 | 0.279 | 0.238 |
| 72 | 0.278 | 0.277 | 0.279 | 0.275 | 0.276 | 0.241 |
| 86 | 0.280 | 0.277 | 0.281 | 0.279 | 0.280 | 0.238 |

### Example 4: Color Development in Aging Samples

The colors of the samples were correlated to the Sherwin-Williams standard color wheel (CS 1/09) during the course of aging. This color set was used as a guide to note the onset of color and any relative changes in shade and intensity. For the samples stored conducted under nitrogen (i.e., Example 1), neither the treated nor untreated TCPE developed any color during the 30 weeks at 40° C. In addition, the TCPE exposed to Plasite-coated coupon, both treated and untreated, remained colorless for the duration of the experiment. The untreated TCPE exposed to stainless steel did develop some coloration at week 8 with the appearance of a light yellow-brown tint. At week 10, the color had intensified only slightly. By week 12, the color had turned to a darker brown yellow which continues to darken even further slowly through week 24. The material turned dull brown at week 26 and continues to lighten to pale brown by week 30. Note that the samples from Example 3 followed the same color generation and change pattern. The last of the samples aged under nitrogen, run 19, with untreated TCPE exposed to carbon steel developed a very pale off-white (brown) tint at week 10 of aging at 40° C.

The untreated TCPE samples aged in air (Example 2) also showed some coloration with time. For the material exposed to Plasite, run 14, a light yellow coloration appeared at week 24, which transitioned to yellow green then green yellow over the subsequent 4 weeks and finally changed to a very pale yellow by week 30. The material exposed to carbon steel, run 21, showed a very pale yellow-green tint at week 10.

The untreated TCPE which had been modified with THYMOL stabilizer and stored under nitrogen or in air all maintained the coloration due to the presence of the stabilizer. These colors ranged from light red-yellow to darker yellow orange over the course of the 30 week experiment and seemed somewhat independent of exposure to coupons. Of course, the higher THYMOL concentrations 20 ppmw vs 10 ppmw) endowed a darker color to the solution. Curiously, the treated TCPE material did not develop any coloration when THYMOL was added indicating that the color developed in the untreated runs was a result of the interaction of the THYMOL with an impurity removed by alumina (run 12 and run 13).

1. A process for stabilizing a chloropropene, the process comprising
a) purging a system containing the chloropropene with an inert gas; and
b) adding a stabilizing amount of at least one substituted phenol to the chloropropene, the stabilizing amount is from about 1 ppm to about 10,000 ppm by weight of the chloropropene;
wherein the chloropropene is substantially free of water and/or oxygenates; and
wherein the inert gas is nitrogen, argon, helium, or mixtures thereof.

2. The process of claim 1, wherein the chloropropene is contacted with an absorbent, an alkylene oxide, or combinations thereof to remove water; and
wherein the absorbent is a molecular sieve, alumina, active carbon, clay, silica gel, zeolite, or a combination thereof.

3. The process of claim 1, wherein the substituted phenol comprises at least one substituent chosen from chloro, nitro, nitroso, alkoxy, hydroxy, amino, alkylamino, keto, alkanoyl, alkyl, substituted alkyl, alkenyl, substituted alkenyl, aryl, or substituted aryl.

4. The process of claim 1, wherein the substituted phenol is chosen from 2-isopropyl-5-methylphenol, p-methoxyphenol, p-tert-amylphenol, p-sec-butylphenol, 2,4-dimethyl-6-tert-butylphenol, 2-methoxyhydroquinone, butylated hydroxy anisole, butylated hydroxy toluene, or a combination thereof.

5. The process of claim 1, wherein the substituted phenol is 2-isopropyl-5-methylphenol.

6. The process of claim 1, wherein the system is in a container.

7. The process of claim 6, wherein the container is a metal container lined with a polymeric coating;
wherein the metal comprises stainless steel, carbon steel, or a monel metal; and
wherein the polymeric coating is a phenolic or epoxy coating.

8. The process of claim 1, wherein the process further comprises padding the system with an inert gas and sealing the system,

9. The process of claim 1, wherein the process further comprises transferring the chloropropene from the system to a storage container, padding the storage container with an inert gas, and sealing the storage container.

10. The process of claim 8, wherein the inert gas is nitrogen.

11. The process of claim 1, wherein the chloropropene is stable for at least six months at a temperature less than or equal to about 40° C.

12. The process of claim 1, wherein the chloropropene is 1,1,2,3,-tetrachloropropene and the substituted phenol is 2-isopropyl-5-methylphenol.

13. A composition comprising a tetrachloropropene and a stabilizing amount of 2-isopropyl-5-methylphenol.

14. The composition of claim 13, wherein the tetrachloropropene is 1,1,2,3,-tetrachloropropene.

15. The composition of claim 13, wherein the stabilizing amount of 2-isopropyl-5-methylphenol is from about 1 ppm to about 10,000 ppm by weight of the tetrachloropropene.

16. The composition of claim 13, which is substantially free of oxygen.

17. The composition of claim 13, which is substantially free of water and/or oxygenates.

18. A container which comprises the composition of claim 13.

19. The container of claim 18, wherein the container is a metal container lined with a polymeric coating;
wherein the metal comprises stainless steel, carbon steel, or a nickel alloy; and
wherein the polymeric coating is a phenolic or epoxy coating.

20. The container of claim 13, wherein the container comprises a pad of inert gas and the container is sealed.
